(19) United States
(12) Patent Application Publication
(10) Pub. No.: US 2013/0331292 A1
(43) Pub. Date: Dec. 12, 2013

(54) SCREENING METHOD

(71) Applicant: Karl Andersson, Uppsala (SE)

(72) Inventor: Karl Andersson, Uppsala (SE)

(73) Assignee: GE HEALTHCARE BIO-SCIENCES AB, UPPSALA (SE)

(21) Appl. No.: 14/000,431

(22) PCT Filed: Feb. 28, 2012
(86) PCT No.: PCT/SE12/50220

§ 371 Date: Aug. 20, 2013

(30) Foreign Application Priority Data
Feb. 28, 2011 (SE) 1150183-0

(51) Int. Cl. G01N 33/543

(52) U.S. Cl. CPC G01N 33/543
USPC 506/9; 506/39

(57) ABSTRACT

A method of screening a plurality of fluid samples for the presence of analytes capable of specifically binding to a ligand immobilized on a sensing surface of a sensor, wherein respective response curves representing the progress of each interaction with time are produced, comprises subjecting a set of resulting response curves to an evaluation procedure comprising determining for each response curve a binder classification based on at least two binding-related features of the response curve, identifying response curves for which the binder classification deviates significantly from that of the remaining response curves as a group, and classifying these deviating response curves as representing sample analytes which are binding partners to the ligand.

### TECHNICAL FIELD OF THE INVENTION

The present invention relates to a method of screening a pool of molecular species for capability of specifically binding to a desired receptor or ligand, particularly screening of molecular libraries, such as drug libraries.

### BACKGROUND OF THE INVENTION

Optical biosensors based on surface plasmon resonance (SPR) are today widely used for analyzing a wide range of biological and chemical interactions. SPR biosensors allow the determination of the affinity and kinetics of molecular interactions in real time without the need for a molecular tag or label. Analytes in a solution are contacted with a sensing surface with immobilized binding partner, or ligand. Binding of analyte to a surface-bound binding partner alters the refractive index at the sensing surface, and this refractive index change can be monitored to measure accurately the amount of bound analyte, its affinity for the receptor and the association and dissociation kinetics of the interaction. Commercial SPR biosensor systems are available which permit a high degree of automation and parallelization, and may therefore be used for high throughput screening assays.

A typical screening procedure, wherein a plurality of fluid samples are tested for the presence of species capable of specifically binding to a desired receptor or ligand comprises contacting each sample with a sensing surface supporting the receptor or ligand and usually also a reference surface without immobilized receptor or ligand, measuring the responses at the surfaces, and evaluating the measurement data including subtracting the response at the reference surface from that at the sensing surface.

Typically, the binding data obtained are first subjected to a number of signal correction adjustments, including at least some of molecular weight adjustment to compensate response values for different analyte sizes, bulk refractive index surface errors, adjustment for decreasing activity of the immobilized ligand (protein), and capture level adjustment when different ligands are used. A binding level against cycle number (i.e. sample no.) is plotted and a binding level limit (or sometimes more than one limit) is then selected by the user, all samples (analytes) exhibiting a binding level above the selected limit being considered as more or less strong binders.

While this procedure is simple and in several respects gives comprehensible results, the user risks missing true binders which for some reason do not reach the correct level. This could, for example, be the case when screening a sample library with different and potentially unknown sample concentrations. A strong binder present at a low concentration would then give a signal which is below the selected level or threshold, in spite of the fact that it appears from the binding curve that the substance is a binder.

A more sophisticated screening evaluation method which identifies all true binders would therefore be desired. It is an object of the present invention to provide such a method.

### SUMMARY OF THE INVENTION

The above-mentioned object as well as other objects and advantages are achieved by an evaluation method based on a multiparametric analysis of the response curves, or binding curves, rather than merely measurement of binding levels therefrom. In brief, the present invention is based on determining from the shape or appearance of the curve or a part or parts thereof the binding capability of the analyte represented by the response curve.

The present invention therefore, in one aspect thereof, provides a method of screening a plurality of fluid samples for the presence of analytes capable of specifically binding to a ligand immobilized on a sensing surface of a sensor, wherein respective response curves representing the progress of each interaction with time are produced. The method is characterized in that it comprises subjecting a set of resulting response curves to an evaluation procedure comprising determining for each response curve a binder classification based on at least two binding-related features of the response curve and identifying response curves for which the binder classification deviates significantly from that of the remaining response curves as a group. These identified deviating response curves are then classified as representing sample analytes which are binding partners to the ligand.

In a preferred embodiment, the evaluation procedure comprises the steps of:
a) selecting at least two binding-related features for the response curves, and for each different binding-related feature defining at least one binding-descriptor,
b) determining for each response curve in the set thereof, values for the different binding-descriptors,
c) based on the values for the different binding-descriptors, computing for each response curve a binder classification (e.g. a binder classification measure) representing the binding character of that response curve in relation to the average binding character of all response curves of the set,
d) selecting response curves having binder classifications deviating significantly (e.g. by at least a predetermined amount) from those of the remaining response curves, and identifying these curves as representing analytes which are binders.

Other preferred embodiments are set forth in the dependent claims.

In another aspect, the present invention provides an analytical system for studying molecular interactions, which comprises data processing means for classifying each response curve with regard to binding capability of the analyte represented by the response curve.

In still another aspect, the present invention provides a computer program product comprising program code means for performing the response curve evaluation procedure as defined for the method aspect above.

In yet another aspect, the present invention provides a computer program product comprising program code means stored on a computer readable medium for performing the response curve evaluation procedure as defined for the method aspect above.

A more complete understanding of the present invention, as well as further features and advantages thereof, will be obtained by reference to the following detailed description and the accompanying drawings.

### BRIEF DESCRIPTION OF THE DRAWINGS

FIG. 1 is a sensorgram showing the interaction between a sample and a target molecule.

FIG. 2 is a flow chart showing the steps in an exemplary embodiment of the method of the present invention.

### DETAILED DESCRIPTION OF THE INVENTION

Unless defined otherwise, all technical and scientific terms used herein have the same meaning as commonly understood by a person skilled in the art related to this invention. Also, the singular forms “a”, “an”, and “the” are meant to include plural reference unless it is stated otherwise

As mentioned above, the present invention provides an improvement in screening assays, such as the screening of drug libraries and the like for compounds having a desired binding specificity. In brief, the invention is based on producing response curves (binding curves) for the interaction of analyte compounds and a receptor or ligand immobilized on sensor surface, and determining from the resulting binding curves, rather than values for binding levels, features of the curve shape or appearance which reveal a sign or tendency of binding, assigning a measure or measures to these features, and classifying an analyte as a binder when the determined measure or measures for the analyte deviates substantially from those of the rest of the analytes (the term “measure” being to be understood in a broad sense herein).

Before describing the invention any further, however, the concept of biosensors, and especially SPR sensors, will be briefly described.

A biosensor is typically based on label-free techniques, detecting a change in a property of a sensor surface, such as mass, refractive index or thickness of the immobilized layer. Typical sensors for the purposes of the present invention include, but are not limited to, mass detection methods, such as optical methods and piezoelectric or acoustic wave methods, including e.g. surface acoustic wave (SAW) and quartz crystal microbalance (QCM) methods. Representative optical detection methods include those that detect mass surface concentration, such as reflection-optical methods, including both external and internal reflection methods, which may be angle, wavelength, polarization, or phase resolved, for example evanescent wave ellipsometry and evanescent wave spectroscopy (EWS, or Internal Reflection Spectroscopy), both of which may include evanescent field enhancement via surface plasmon resonance (SPR), Brewster angle refractometry, critical angle refractometry, frustrated total reflection (FTR), scattered total internal reflection (STIR) (which may include scatter enhancing labels), optical wave guide sensors, external reflection imaging, evanescent wave-based imaging such as critical angle resolved imaging, Brewster angle resolved imaging, SPR-angle resolved imaging, and the like. Further, photometric and imaging/microscopy methods, “per se” or combined with reflection methods, based on, for example, surface enhanced Raman spectroscopy (SERS), surface enhanced resonance Raman spectroscopy (SERRS), evanescent wave fluorescence (TIRF) and phosphorescence may be mentioned, as well as waveguide interferometers, waveguide leaking mode spectroscopy, reflective interference spectroscopy (RIfS), transmission interferometry, holographic spectroscopy, and atomic force microscopy (AFR).

Among the biosensors mentioned above may especially be mentioned optical evanescent wave-based sensors including surface plasmon resonance (SPR) sensors, frustrated total reflection (FTR) sensors, and waveguide sensors, especially SPR-biosensors.

Several SPR based biosensor systems are commercially available today. Exemplary such SPR-biosensors include the flow-through-cell-based Biacore® systems (GE Healthcare, Uppsala, Sweden) and ProteOn™ XPR system (Bio-Rad Laboratories, Hercules, Calif., USA) which use surface plasmon resonance for detecting binding interactions between molecules, “analytes”, in a sample and molecular structures, “ligands”, immobilized on one or more sensing surfaces or spots.

The phenomenon of surface plasmon resonance, or SPR, is well known. Suffice it to say that SPR arises when light is reflected under certain conditions at the interface between two media of different refractive indices, and the interface is coated by a metal film, typically silver or gold. In the Biacore® system, the two media are the sample and the glass of a sensing surface provided by a sensor chip which is contacted with the sample through a microfluidic flow system. The metal film is a thin layer of gold on the chip surface supporting a ligand for an analyte in the sample. SPR causes a reduction in the intensity of the reflected light at a specific angle of reflection. This angle of minimum reflected light intensity, the “SPR angle”, varies with the refractive index close to the surface on the side opposite from the reflected light, in the Biacore® system the sample side. As analyte in a sample solution contacted with the chip surface binds to the immobilized ligand, the refractive index near the chip surface increases, leading to a shift in the SPR angle. When the sample solution is replaced by a solution without analyte, the analyte-ligand complex dissociates and the refractive index decreases, resulting in the SPR angle shifting back.

As sample is passed over the sensing surface, the progress of binding of analyte to immobilized ligand, as detected by the shift in SPR angle, directly reflects the rate at which the interaction occurs. Injection of sample is usually followed by a buffer flow during which the detector response reflects the rate of dissociation of the complex on the surface. A typical output from the system is a graph or curve describing the progress of the molecular interaction with time, including an association phase part and a dissociation phase part. This binding curve recording the shift in the SPR angle as a function of time, and which is usually displayed on a computer screen, is often referred to as a “sensorgram”. The angular shift is measured in response units (RU), 1 RU being equal to a 10⁻⁶ change in refractive index. Usually, the sample also passes a reference surface without immobilized ligand for referencing away non-specific binding events and other effects unrelated to specific binding.

With the Biacore® and analogous SPR-based sensor systems it is thus possible to determine in real time without the use of labeling, and often without purification of the substances involved, not only the presence and concentration of a particular molecule, or analyte, in a sample, but also additional interaction parameters, including kinetic rate constants for association (binding) and dissociation in the molecular interaction as well as the affinity for the surface interaction.

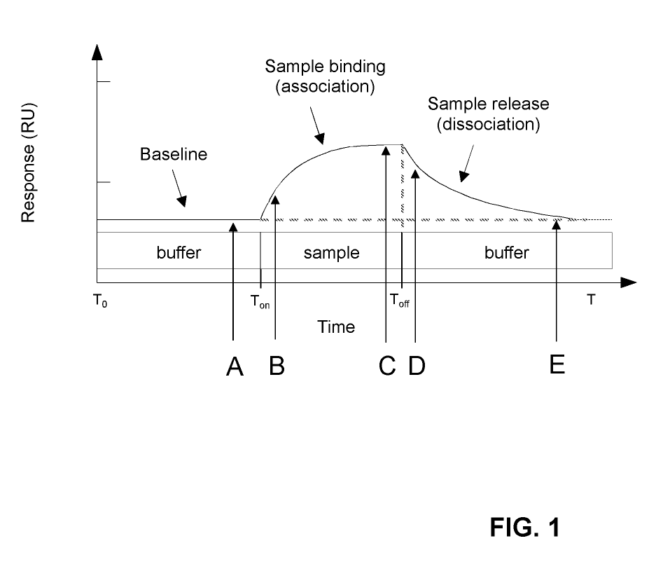

A representative sensorgram for the Biacore® instrument is shown in FIG. 1, which depicts a sensing surface having an immobilized ligand (e. g. an antibody) interacting with analyte in a sample. The y-axis indicates the response (here in resonance units (RU)) and the x-axis indicates the time. Initially, buffer is passed over the sensing surface giving the “baseline response” in the sensorgram. Starting at Tₒₙ the sample is injected over the sensing surface. During sample injection, an increase in signal is observed due to binding of the analyte (i. e. association) to a steady state condition where the resonance signal plateaus. At the end of sample injection T_{off}, the sample is replaced with a continuous flow of buffer and a decrease in signal reflects the dissociation, or release, of analyte from the surface. The slope of the association/dissociation curves provides valuable information regarding the interaction kinetics, and the height of the resonance signal represents surface concentration (i. e. the response resulting from an interaction is related to the change in mass concentration on the surface). The detection curves, or sensorgrams, produced by biosensor systems based on other detection principles will have a similar appearance.

Sometimes the sensorgrams produced may for various reasons be of unacceptable quality and therefore have to be discarded. The quality of sensorgrams is normally done by the user making an overlay plot of the curves to be analyzed and visually searching for oddities in the curves. However, when large sets of sensorgrams are produced, it will be impracticable for the user to inspect all the sensorgrams one at a time for assessing the quality thereof.

A procedure, or rather an algorithm, for an at least semi-automated quality assessment and classification of sensorgrams and removal of bad sensorgrams is disclosed in WO 03/081245 A1 (the full disclosure of which is incorporated by reference herein). In this procedure, the response curves, specifically sensorgrams, are subjected to a quality assessment which comprises representing the response curves with one or more quality descriptors, applying a quality classification method to the descriptors to find outliers, and removing the outliers. More particularly, a quality measure or classification is first calculated for each individual sensorgram. The majority of the sensorgram curves are then assumed to be “good”, and the response curves having deviating quality classifications, preferably in the form of the “statistical distance” of the curve (in a quality measure sense) to the total curve amount are selected. The selected curves are then subjected to a validation procedure which may include visual inspection of the sensorgrams.

### The Invention

The present invention is based on the idea of using a similar approach as the response curve quality assessment described above for the screening a collection or library of species or compounds (analytes), e.g. a molecular library, such as a drug library (typically a small molecule library, such as a fragment library), for the binding to a desired receptor or ligand. More specifically, a binder classification based on at least two binding-related features of the response curve is determined for each response curve and, assuming that the majority of the analytes to be screened are non-binders or bad binders, true binders may be identified by the “outliers”, i.e. the response curves whose binder classification deviate most from the “average” binder classification of the total amount of curves.

Preferably, a threshold or limit is set beyond which all objects are classified as binders.

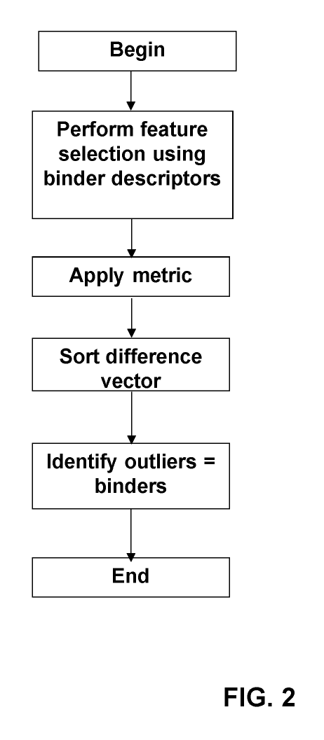

A flow chart of an embodiment of an algorithm for such a screening evaluation of the response curves is shown in FIG. 2 and will be described below.

To determine the binder classification mentioned above, at least two binding-related features in the form of response curve (sensorgram) features or parameters which reveal a tendency to binding are first selected. A few examples of such binding-related features will now be disclosed with reference to FIG. 1:
As mentioned in the introduction one such feature is a high response level during the end of the association phase, e.g. at C in FIG. 1 compared to the base line level before injection A which reveals that a large amount of the sample analyte has bonded to the sensing surface.
High response level at B short after Tₒₙ, or high slope at B, thus indicating that fast initial interaction.
Low slope at C short before T_{off}, as a non-flattened out steady state may indicate non-specific binding and slow interaction.
High slope at D short after T_{off}, indicating reversible interaction, and a compound which shows a rapid off-rate is often a binder
High response level at E after dissociation phase, indicating irreversible binding etc.

Other binding-related features may be of more relative type and may e.g. relate to identifying characteristic kinetic behaviours:
, A binding-like curvature during the association phase (e.g. the response from Tₒₙ to T_{off}, fitted against a theoretical model),
A distinct dissociation phase, and potentially showing a nice exponential 1:1 dissociation phase (e.g by fitting the response to a theoretical model)
   Just to mention a few. Other suitable binding-related features may readily be selected by the skilled person.

While only a small number of binding-related features may be used to determine the binder classification, a greater number of features may be used, e.g. five or more different features. According to one embodiment of the present invention, the binder classification may be determined using binding-related features that are essentially independent of each other to enable capture of binders that otherwise would have been considered as non-binders.

Based on the selected binding-related features one or more descriptor is defined, a descriptor being a formula or algorithm which with the binding-related feature as input produces, for example, a numerical value as output. If, for instance, one of the binding-related features is high slope at B, the corresponding descriptor may be a function that produces a weighted slope value as output, or it may be a threshold function that produces a fixed value for all slopes exceeding one or more predefined threshold values and other value for slopes lower than the threshold values. The descriptor function should be selected according to the type of binding interactions that should be classified. E.g a sensorgram for which the high slope at B descriptor produces a value has a value of 10 indicates a more binding like behaviour than a sensogram with a high slope at B descriptor with a value of 5. A descriptor reporting the high response level at C is in its simplest form only a weighted relative response (the response at the end of a sample injection relative to the baseline level). For descriptors related to relative binding-related features such as binding-like curvature during the association phase, may e.g be based on a residual from a fitting process and/or a look up table providing predefined descriptor values depending on the deviance from an ideal curvature or the like. An example of a descriptor table (matrix) is given in Table 1 below.

TABLE 1

| | | | Binding-like |
|---|---|---|---|
| | | High | curvature |
| | High | response | during |
| Cycle | slope at B | level at C | association |
| 1 | 2.0 | 8.2 | 2 |
| 2 | 0.3 | 4.8 | 6 |
| 3 | 4.2 | 6.1 | 7 |
| 4 | 1.6 | 5.1 | 1 |
| 5 | 9.8 | 2.0 | 9 |

The descriptors of each individual response curve, or sensorgram, are then transformed to a vector of descriptor values. In this way each sensorgram has been reduced to a set of descriptor values representing the different binding-related features. Thus, instead of the sensorgram, there are now a small number of figures in a vector which describe only the properties of interest of the sensorgram. The descriptor vectors for all the sensorgrams in the set are collected in a descriptor matrix.

A binding classification metric, usually an equation, is then applied to the descriptor matrix to estimate the difference in analyte binding capability between each sensorgram and the rest of the sensorgrams in the set. This translates the descriptor matrix to a difference vector (containing differences) and validation matrix (containing estimates of the contribution to the difference of each descriptor).

The difference vector is then sorted with regard to difference magnitude to obtain a sorted difference vector and validation matrix. Sensorgrams with large differences will then be “outliers” with respect to the binding-descriptors and thus represent binders. When a binder metric equation is used as described above, each vector may be seen as a point in space, and the similarity (or difference) between sensorgrams may then be represented by the distances between the respective points.

To measure the distances between the descriptor vectors, a statistical method may be used which measures the distance from each respective vector to all the other vectors seen as a group. Thereby each vector is reduced to a single value that describes how similar the descriptor vector is to all the other vectors. Sensorgrams having approximately the same value are then about equal “binding-wise” regarding the descriptors and the statistical method. Statistical methods that may be used include methods that are per se well known to the skilled person. Some specific exemplary methods are briefly described below.

The “Euclidian distance” between two points is the length of the line segment connecting them.

“Mahalanobis distance” is a generalisation of the Euclidian distance between two points. Areas with a constant distance are ellipsoids centered around the mean value. When the descriptors are uncorrelated and the variances are equal to one in all directions, the areas are spheres and the Mahalanobis distance is equivalent to the Euclidian distance. The measure as such comprises a normalization of the descriptors by means of the inverse of the covariance matrix.

“Manhattan distance” sums up the descriptor vector.

The binder classification may include rescaling, or “normalizing”, the descriptor values to make them comparable. An exemplary normalization method is the “mean centre” method, which sets the mean value of the descriptor values to zero. Other examples of normalization procedures are “mean centre and unit variance” (sets the mean value of the descriptors to zero and variance to one), and “unit variance” (sets variances to one).

Alternative specific methods, according to the invention, for binder classification based on the descriptor vectors described above include, but are not limited to, Artificial Neuron Networks (ANN), hierarchical clustering, k-means clustering, Self Organizing Maps (SOM) and Support Vector Machines (SVM) representing both supervised and non-supervised learning approaches applicable to the task. Such alternative methods are per se well-known to a person skilled in the art.

The method steps according to the algorithm outlined above may conveniently be implemented by software run on an electrical data processing device, such as a computer. Such software may be provided to the computer on any suitable computer-readable medium, including a record medium, a read-only memory, or an electrical or optical signal which may be conveyed via electrical or optical cable or by radio or other means.

The present invention is not limited to the above-described preferred embodiments. Various alternatives, modifications and equivalents may be used. Therefore, the above embodiments should not be taken as limiting the scope of the invention, which is defined by the appending claims.

What is claimed is:

1. A method of screening a plurality of fluid samples for the presence of analytes capable of specifically binding to a ligand immobilized on a sensing surface of a sensor, wherein respective response curves representing the progress of each interaction with time are produced, which method comprises subjecting a set of resulting response curves to an evaluation procedure comprising determining for each response curve a binder classification based on at least two binding-related features of the response curve, identifying response curves for which the binder classification deviates significantly from that of the remaining response curves as a group, and classifying these deviating response curves as representing sample analytes which are binding partners to the ligand.

2. The method of claim 1, wherein the evaluation procedure comprises the steps of:
a) selecting at least two binding-related features for the response curves, and for each different binding-related feature defining at least one binding-descriptor,
b) determining for each response curve in the set thereof, values for the different binding-descriptors,
c) based on the values for the different binding-descriptors, computing for each response curve a binder classification representing the binding character of that response curve in relation to the average binding character of all response curves of the set,
d) selecting response curves having binder classifications deviating significantly from those of the remaining response curves, and identifying these curves as representing analytes which are binders.

3. The method of claim 2 wherein step d) comprises selecting response curves having binder classifications deviating by at least a predetermined amount from those of the remaining response curves.

4. The method of claim 1 wherein the descriptor values are normalized.

5. The method of claim 2 wherein step b) of claim 2 further comprises transforming the binding-descriptor values for each response curve to a binding-descriptor vector.

6. The method of claim 5, wherein a binding-descriptor matrix is created from the binding-descriptor vectors.

7. The method of claim 2, wherein computing a binder classification in step c) of claim 2 comprises determining for each binding descriptor vector the difference between the vector and the rest of the binding descriptor vectors in the set of response curves as a group.

8. The method of claim 7, wherein determining the differences between the vectors comprises determining a statistical measure of the distance from each binding descriptor vector to the rest of the binding descriptor vectors as a group.

9. The method of claim 7, wherein a difference vector is created from the computed differences.

10. The method of claim 5, wherein the evaluation procedure comprises using a method selected from Artificial Neuron Networks (ANN), hierarchical clustering, k-means clustering, Self Organizing Maps (SOM) and Support Vector Machines (SVM).

11. The method of claim 1, wherein the at least one binding-related feature or parameter is selected from a high signal level of the response curve during the end part of an association phase, a binding-indicating curvature of the response curve during an association phase, and a distinct dissociation phase.

12. An analytical system for detecting molecular binding interactions, comprising:
(i) a sensor device comprising at least one sensing surface, detection means for detecting molecular interactions at the at least one sensing surface, and means for producing response curves representing the progress of each interaction with time, and
(ii) data processing means for performing the response curve evaluation procedure as defined in claim 1.

13. A computer program product comprising program code means for performing the response curve evaluation procedure as defined in claim 1.

14. A computer program product comprising program code means stored on a computer readable medium for performing the response curve evaluation procedure as defined in claim 1.
